(19) United States
(12) Patent Application Publication
(10) Pub. No.: US 2011/0020217 A1
(43) Pub. Date: Jan. 27, 2011

(54) TREATMENT OF MELANOMA

(75) Inventors: Malcolm Philip Young, Newcastle Upon Tyne (GB); Catherine Mary Thomas, Newcastle Upon Tyne (GB); Olusola Clement Idowu, Newcastle Upon Tyne (GB)

Correspondence Address: K&L Gates LLP, STATE STREET FINANCIAL CENTER, One Lincoln Street, BOSTON, MA, 02111-2950, US

(73) Assignee: E-THERAPEUTICS PLC, Tyne& Wear (GB)

(21) Appl. No.: 12/738,664

(22) PCT Filed: Oct. 10, 2008
(86) PCT No.: PCT/GB2008/003415

§ 371 Date: Oct. 4, 2010

(30) Foreign Application Priority Data
Oct. 10, 2007 (GB) 0719771.8

(51) Int. Cl. A61K 31/352; A61K 31/496; A61K 31/517; A61K 31/5355; A61K 31/44; A61K 31/404; A61K 51/00; A61K 31/4184; A61P 35/00; A61P 35/04

(52) U.S. Cl. 424/1.11; 514/454; 514/252.19; 514/266.3; 514/234.5; 514/252.18; 514/266.24; 514/346; 514/414; 514/44 R; 514/394

(57) ABSTRACT

There is described a pharmaceutical composition comprising dexanabinol, or a derivative thereof, in combination with a second therapeutic agent that targets BRAF or MEK, and a pharmaceutically acceptable adjuvant, diluent or carrier. There is also described a method of treating a patient suffering from melanoma and uses related thereto.

### FIELD OF THE INVENTION

The present invention provides combination medicaments and methods for the treatment of melanoma.

### BACKGROUND

Incidence of melanoma cases has doubled every year since the 1940s. Melanoma is now the sixth most common cancer in men, and the seventh most common cancer in women. Its incidence is increasing in all parts of the world (Parker, S et al, 1997). The 5 year survival rate for melanoma is 30 to 40%, with malignant melanoma carrying the highest risk of mortality from metastasis (Jemal et al, 2001); Spread of the disease to distant organs such as liver, bone and brain, reduces the 5 year survival to less than 12%. There is currently no effective long-term treatment for patients with metastatic (Stage 1V) melanoma. Standard chemotherapy regimens do not impart a significant long term survival benefit in these patients, and chemotherapy may be associated with a degree of morbidity due to toxicity. There is an obvious need to develop new targeted therapies for melanoma, both to prevent cancer progression and to treat advanced disease.

Finding new effective treatments for melanoma has proved very challenging. High resistance to conventional chemotherapeutic agents and radiation is a hallmark melanoma. (Smalley and Eisen, 2003; Strauss et al., 2003). Research has now shifted to identifying melanoma gene mutations and the associated perturbations of signal transduction pathways, in the hope that more specific targeted therapies can be developed. Several cellular pathways important to cell proliferation, apoptosis and resistance or metastases have been shown to be activated in melanoma. Melanoma likely develops multiple defects, including loss of regulatory functions or the gain of anti-apoptotic or proliferative functions. Thus, therapeutic agents that could inhibit several signalling pathways simultaneously would be highly desirable. In addition, administration of standard chemotherapies in combination with new agents may afford the traditional chemotherapies a new lease of life in the melanoma context, if chemo resistance is inhibited. The challenge now is to develop selective agents to target these aberrant pathways.

An important signalling pathway in melanoma is the RAS/RAF mitogen-activated protein kinase (MAPK)/extracellular signal-regulated kinase (ERK) kinase (MEK)/ERK cascade. This signalling pathway leads to the phosphorylation of several cytosolic and nuclear proteins to regulate gene expression, and thus plays a critical role in cell proliferation, differentiation, senescence and survival. One of the three RAF isoforms in humans, BRAF, is mutated in 50% to 70% of melanoma cases. The ERK pathway is therefore hyperactivated in most melanoma cases. A number of small molecule inhibitors that target BRAF, or its downstream effector, MEK, are in clinical and preclinical development. Sorafenib for example, is a broad specificity kinase inhibitor that targets BRAF, CRAF and receptor tyrosine kinases. Sorafenib is 4-[4-[[4-chloro-3-(trifluoromethyl)phenyl]carbamoylamino]phenoxy]-N-methyl-pyridine-2-carboxamide and is described in U.S. Pat. No. 7,235,576. When used as a monotherapy, sorafenib only shows marginal clinical benefit in melanoma patients. One hypothesis put forward to explain the poor performance of sorafenib and other BRAF inhibitors clinically is the pro-survival influence of tumour necrosis factor alpha (TNF-alpha). Inflammation plays an important role in the tumour microenvironment and a number of studies have shown infiltration of TNF-alpha secreting immune cells into melanomas. Increased cancer severity has also been correlated with polymorphisms that elevate TNF-alpha expression. Combining drugs that target BRAF or MEK with an agent that inhibits the rescue pathways stimulated by TNF-alpha provides a rational approach to treating melanoma. We have previously described a novel treatment for melanoma using dexanabinol in our co-pending UK patent application No. GB0713116.2). In addition to its utility as a melanoma monotherapy, dexanabinol would be a highly suitable agent to combine with anti-BRAF or anti-MEK therapies. Not only does dexanabinol target NFκB, an important antiapoptotic and proinflammatory target in melanoma, but it also blocks the production of TNF-alpha at a post-translational level. Dexanabinol thus targets both the key proteins that act as a barrier to successful anti-BRAF and anti-MEK therapies.

### SUMMARY OF THE INVENTION

We have now found a method of increasing the therapeutic effectiveness of agents that target BRAF and MEK in melanoma, by combining the agents with dexanabinol.

Therefore, in accordance with a first aspect, the present invention provides a pharmaceutical composition comprising dexanabinol, or a derivative thereof, in combination with a second therapeutic agent that targets BRAF or MEK, and a pharmaceutically acceptable adjuvant, diluent or carrier.

The treatment of melanoma according to the invention may comprise, separately, simultaneously or sequentially inhibiting NFκB and TNF-alpha activity in a melanoma cancer cell by providing to the cell dexanabinol or a derivative thereof, in combination with an anti-BRAF or anti-MEK agent.

Thus, according to a further aspect of the invention we provide dexanabinol, or a derivative thereof, separately, simultaneously or sequentially in combination with a second therapeutic agent that targets BRAF or MEK for the treatment of melanoma.

Thus, the second therapeutic agent may target BRAF. Alternatively, the second therapeutic agent may target MEK.

Examples of therapeutic agents that target BRAF include, dasatinib, erlotinib, gefitinib, imatinib, lapatinib, sorafenib and sunitinib, or a derivative thereof. Preferentially, the derivative of the BRAF inhibitor may be a salt. Thus, according to the invention the BRAF inhibitor may be selected from the group consisting of dasatinib, erlotinib hydrochloride, gefitinib, imatinib mesilate, lapatinib, sorafenib tosylate and sunitinib malate. Preferably the BRAF inhibitor is sorafenib tosylate.

Thus, according to the invention the MEK inhibitor may be selected from the group consisting of certain experimental compounds, some of which are currently in Phase 1 or Phase II studies, namely PD-325901 (Phase 1), XL518 (Phase 1), PD-184352, PD-318088, AZD6244 (Phase II) and CI-1040.

Alternatively, the treatment of melanoma may comprise the inhibition of tumorigenesis of a melanoma cancer cell by contacting the cell with an effective amount of dexanabinol or a derivative thereof and either an anti-BRAF or anti-MEK agent. Inhibition of tumorigenesis includes inducing both cytotoxicity and apoptosis in the cancer cell.

Dexanabinol, or a derivative thereof in combination with either an anti-BRAF or anti-MEK agent for the treatment of melanoma is advantageous, inter alia, because it shows reduced toxicity, reduced side effects and/or reduced resistance when compared to currently employed chemotherapeutic agents.

It is further contemplated that the anti-BRAF or anti-MEK agent may be administered with the dexanabinol, or a salt or a derivative thereof, separately, simultaneously or sequentially.

The term “derivative” used herein shall include any conventionally known derivatives of dexanabinol, such as, inter alia, solvates. It may be convenient or desirable to prepare, purify, and/or handle a corresponding solvate of the compound described herein, which may be used in any one of the uses/methods described. The term solvate is used herein to refer to a complex of solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

According to a further aspect of the invention we provide a method of treatment or alleviation of melanoma which comprises contacting a melanoma cell with a therapeutically effective amount of dexanabinol, or a derivative thereof, separately, simultaneously or sequentially in combination with an effective amount of a second therapeutic agent capable of targeting BRAF or MEK.

Thus, the second therapeutic agent may target BRAF. Alternatively, the second therapeutic agent may target MEK.

Accordingly, and in one embodiment, the present invention provides a use of dexanabinol and/or a derivative thereof in combination with either an anti-BRAF or anti-MEK agent in the manufacture of a medicament for the treatment of melanoma.

Furthermore, in a second aspect, the present invention provides a method of treatment melanoma, said method comprising the administration of a therapeutically effective amount of dexanabinol and derivatives and/or combinations with anti-BRAF or anti-MEK agents.

We further provide the use of a BRAF inhibitor in the manufacture of a combination therapy with dexanabinol, for the treatment or alleviation of melanoma. In addition, we provide a BRAF inhibitor, separately, simultaneously or sequentially in combination with dexanabinol for the treatment of melanoma.

In a further aspect of the invention we provide the use of a MEK inhibitor in the manufacture of a combination therapy with dexanabinol, for the treatment or alleviation of melanoma. In addition we provide a MEK inhibitor, separately, simultaneously or sequentially in combination with dexanabinol for the treatment of melanoma.

Dexanabinol and derivatives and combinations of anti-BRAF or anti-MEK agents are known per se and may be prepared using methods known to the person skilled in the art or may be obtained commercially. In particular, dexanabinol and methods for its preparation are disclosed in U.S. Pat. No. 4,876,276.

Advantageously, in the use and or method of the invention the compound and derivatives and combinations of either anti-BRAF agents or anti-MEK agents may be administered orally, or intravenously.

Thus, in the use, method and/or composition of the invention of the compound may be put up as a tablet, capsule, dragee, suppository, suspension, solution, injection, e.g. intravenously, intramuscularly or intraperitoneally, implant, a topical, e.g. transdermal, preparation such as a gel, cream, ointment, aerosol or a polymer system, or an inhalation form, e.g. an aerosol or a powder formulation.

Compositions suitable for oral administration include tablets, capsules, dragees, liquid suspensions, solutions and syrups;

Compositions suitable for topical administration to the skin include creams, e.g. oil-in-water emulsions, water-in-oil emulsions, ointments or gels;

examples of such adjuvants, diluents or carriers are: for tablets and dragees—fillers, e.g. lactose, starch, microcrystalline cellulose, talc and stearic acid; lubricants/glidants, e.g. magnesium stearate and colloidal silicon dioxide; disintegrants, e.g. sodium starch glycolate and sodium carboxymethylcellulose;
for capsules—pregelatinised starch or lactose;
for oral or injectable solutions or enemas—water, glycols, alcohols, glycerine, vegetable oils;
for suppositories—natural or hardened oils or waxes.

It may be possible to administer the compound or derivatives and/or combination thereof or any combined regime as described above, transdermally via, for example, a transdermal delivery device or a suitable vehicle or, e.g. in an ointment base, which may be incorporated into a patch for controlled delivery. Such devices are advantageous, as they may allow a prolonged period of treatment relative to, for example, an oral or intravenous medicament.

Examples of transdermal delivery devices may include, for example, a patch, dressing, bandage or plaster adapted to release a compound or substance through the skin of a patient. A person of skill in the art would be familiar with the materials and techniques which may be used to transdermally deliver a compound or substance and exemplary transdermal delivery devices are provided by GB2185187, U.S. Pat. No. 3,249,109, U.S. Pat. No. 3,598,122, U.S. Pat. No. 4,144,317, U.S. Pat. No. 4,262,003 and U.S. Pat. No. 4,307,717.

1. A pharmaceutical composition comprising dexanabinol, or a derivative thereof, in combination with a second therapeutic agent that targets BRAF or MEK, and a pharmaceutically acceptable adjuvant, diluent or carrier.

2. A pharmaceutical composition according to claim 1 wherein the second therapeutic agent targets BRAF.

3. A pharmaceutical composition according to claim 1 wherein the BRAF inhibitor is selected from the group consisting of dasatinib, erlotinib, gefitinib, imatinib, lapatinib, sorafenib and sunitinib, or a derivative thereof.

4-6. (canceled)

7. A pharmaceutical composition according to claim 1 wherein the second therapeutic agent targets MEK.

8. A pharmaceutical composition according to claim 1 wherein the MEK inhibitor is selected from the group consisting of PD-325901 (Phase 1), XL518 (Phase 1), PD-184352, PD-318088, AZD6244 (Phase II) and CI-1040.

9. Dexanabinol, or a derivative thereof, separately, simultaneously or sequentially in combination with a second therapeutic agent that targets BRAF or MEK for the treatment of melanoma.

10. A method of treatment or alleviation of melanoma which comprises contacting a melanoma cell with a therapeutically effective amount of dexanabinol, or a derivative thereof, separately, simultaneously or sequentially in combination with a second therapeutic agent that targets BRAF or MEK for the treatment of melanoma.

11. A method according to claim 10 wherein the method comprises administration of a pharmaceutical composition comprising dexanabinol, or a derivative thereof, in combination with a second therapeutic agent that targets BRAF or MEK, and a pharmaceutically acceptable adjuvant, diluent or carrier.

12. A method according to claim 10 wherein the second therapeutic agent targets BRAF.

13. A method according to claim 10 wherein the BRAF inhibitor is selected from the group consisting of dasatinib, erlotinib, gefitinib, imatinib, lapatinib, sorafenib and sunitinib, or a derivative thereof.

14-16. (canceled)

17. A method according to claim 10 wherein the second therapeutic agent targets MEK.

18. A method according to claim 10 wherein the second therapeutic agent is selected from the group consisting of PD-325901 (Phase 1), XL518 (Phase 1), PD-184352, PD-318088, AZD6244 (Phase II) and CI-1040.

19. A method according to claim 10 wherein the melanoma cancer cells are premalignant, malignant, metastatic or multidrug-resistant.

20. A method according to claim 10 wherein the treatment of melanoma comprises inhibiting BRAF and/or MEK, separately, simultaneously or sequentially to inhibition of NFκB activity in a melanoma cancer cell.

21. A method according to claim 10 wherein the treatment of melanoma comprises inhibition of tumorigenesis of a melanoma cancer cell.

22. A method according to claim 10 wherein the inhibition of tumorigenesis includes inducing both cytotoxicity and apoptosis in the cancer cell.

23. A method according to claim 10 which comprises administration of a dexanabinol, or a salt or a derivative thereof, in combination with one or more of a therapeutic agent comprising a chemotherapeutic agent, an immunotherapeutic agent, a gene therapy, or a radiotherapeutic agent; separately, simultaneously or sequentially.

24. A method according to claim 10 wherein the therapeutically effective amount of dexanabinol, or a derivative thereof, in combination with a second therapeutic agent that targets BRAF and/or MEK, is administered orally or intravenously.

25. (canceled)

26. (canceled)

27. A BRAF inhibitor, separately, simultaneously or sequentially in combination with dexanabinol for the treatment of melanoma.

28-30. (canceled)
